(19) United States
(12) Patent Application Publication
(10) Pub. No.: US 2024/0041911 A1
(43) Pub. Date: Feb. 8, 2024

(54) USE OF CLADRIBINE FOR TREATING IMMUNE BRAIN DISEASE

(71) Applicant: CHORD THERAPEUTICS SA, GENEVA (CH)

(72) Inventor: KONRAD REJDAK, LUBLIN (PL)

(21) Appl. No.: 18/266,281

(22) PCT Filed: Dec. 6, 2021
(86) PCT No.: PCT/GB2021/053182

§ 371 Date: Jun. 9, 2023

(30) Foreign Application Priority Data
Dec. 10, 2020 (GB) 2019460.1

(51) Int. Cl. A61K 31/7076; A61P 37/02; A61K 9/48

(52) U.S. Cl. CPC A61K 31/7076; A61P 37/02; A61K 9/48

(57) ABSTRACT

2-Chloro-2′-deoxyadenosine, hereinafter referred to as cladribine, or a pharmaceutically acceptable salt thereof may be used in the treatment or amelioration of autoimmune encephalitis, hereinafter referred to as AE, in a patient diagnosed with AE.

### FIELD OF THE INVENTION

The present invention relates to the use of 2-chloro-2′-deoxyadenosine, hereinafter referred to as cladribine, or a pharmaceutically acceptable salt thereof, for treating or ameliorating an autoimmune, brain disorder, in particular the autoimmune brain disorder autoimmune encephalitis, hereinafter referred to as AE.

### BACKGROUND TO THE INVENTION

Autoimmune diseases are a large family of disorders in which the activity of elements of the immune system, which protect against illness by attacking or suppressing infections or malignancies, are aberrantly activated against some of the body's own normal proteins or other structures. In autoimmune brain diseases antibodies that are present in the blood or cerebrospinal fluid of patients bind to target proteins made by cells of the brain, and lead to symptoms that range from moderate to severe. Variability in disease presentation is common and severity can vary markedly between patients with the same disorder.

Encephalitis is a severe inflammatory disorder of the brain with many possible causes and a complex differential diagnosis.

Autoimmune mediated encephalitis is a heterogeneous group of conditions that occur when the body's immune system mistakenly attacks healthy brain cells leading to inflammation of the brain. They are caused by auto-antibodies to various either intra- or extracellular neuronal antigens. AEs can affect all age groups and both genders. They may occur in the context of:
underlying tumour (paraneoplastic),
post-infection
idiopathic (unknown etiology) condition.
   They differ in pathomechanism and pathogenesis, sometimes with strong genetic predisposition and with response to therapy ranging from excellent to negligible.

Patients may experience various neurologic and/or cognitive and psychiatric symptoms, with onset more sudden than typically seen with progressive neurodegenerative conditions such as Alzheimer's and Parkinson's. Symptoms commonly associated with AE include cognitive impairment, memory difficulties, seizures, loss of consciousness, involuntary movements, slowed or loss of ability to speak, and behavioural changes (for example, agitation or loss of inhibition). The onset can be at any age but rarely before young adulthood.

The term “autoimmune encephalitis” generally refers to a family or group of closely related disease processes that share overlapping clinical features and neuroimaging findings but are ultimately differentiated by the specific antibody subtypes driving the underlying immune-mediated attack on different CNS structures. (Kelley et al. Autoimmune Encephalitis: Pathophysiology and Imaging Review of an Overlooked Diagnosis, American Journal of Neuroradiology, June 2017, 38 (6) 1070-1078) The prevalence of autoimmune encephalitis in the USA has been estimated at approximately 14 per 100,000 (Dubey et al; Ann. Neurol.; 83(1); 166-177; (2018)).

In one group of AEs, autoantibodies to extracellular antigens, including extracellular receptors and ion channels, are produced. The binding of antibodies to these extracellular antigens is thought to be pathogenic. This group includes many well-known AE syndromes including anti-NMDA (N-methyl-D-aspartate) receptor encephalitis and anti-LGI1 (leucine rich glioma inactivated-1) antibody encephalitis.

In a second group of AEs autoantibodies to intracellular antigens are found, and the condition is often paraneoplastic with a strong association with a type of cancer.

### Paraneoplastic Syndromes

Paraneoplastic syndromes affecting the CNS are generally thought to develop in cancer when antigens shared by tumor cells and native non-neoplastic neuronal cells result in an antibody-mediated attack on previously immune-privileged neuronal structures. Initially thought to occur in 1% of patients with cancer, more recent data suggest that the true incidence is likely much higher. Paraneoplastic syndromes are most often seen in small-cell lung cancer but can also be seen in a variety of other cancers as well, such as neuroblastoma, germ cell tumor of the testis, breast cancer, Hodgkin lymphoma, thymoma, and immature ovarian teratomas. Regardless of the etiology and antibody profile, there is a clear predilection in autoimmune encephalitis for the presence of antibodies that bind to antigens within the limbic system of the brain. In many cases the underlying tumor cannot be found as paraneoplastic syndrome may precede the detectable growth of cancer or it is a presumed sole manifestation without proven neoplasia.

### Intracellular and Cell-Surface Antigens

In addition to the “paraneoplastic-versus-non-paraneoplastic” categorization, antibody-mediated encephalitic conditions can also be characterized by the type of detectable antibodies, according to the location of their neuronal antigens:
(a) antibodies targeting intracellular antigens and
(b) antibodies targeting antigens on the cell surface, which have or do not have pathogenic activity. This distinction is clinically relevant because it has implications for treatment response, association with an underlying malignancy, and overall long-term prognosis.
   Autoimmune Encephalitis with Intracellular Antigens (AE-IA)

Antibodies in the AE-IA group target intracellular neuronal antigens and are more closely associated with an underlying malignancy, and use the same cytotoxic T-cell mechanisms when targeting the intracellular neuronal antigens and onconeuronal antigens as part of the immune response to cancer. AE-IA Group antibodies are also associated with poor clinical outcomes, characterized by a decreased response to immunomodulatory therapy and an increased prevalence of irreversible neuronal damage, and often have the additional burden of an underlying malignancy.

AE-IA Group antibodies are less specific clinical markers of disease for autoimmune encephalitis and can also be seen in patients with cancer without paraneoplastic syndromes.

Autoimmune Encephalitis with Cell-Surface Antigens (AE-EA)

AE-EA Group antibodies that target cell-surface neuronal antigens are less likely to be associated with an underlying malignancy, and use more “restricted” humoral immune mechanisms of neurotoxicity. AE-EA Group antibodies also represent a more specific clinical marker of disease for antibody-mediated encephalitis, with reduction in serum antibody titers following treatment directly associated with improved neurologic outcomes. AE-EA Group antibodies often target synaptic proteins and can result in the down-regulation of receptors that leads to altered synaptic transmission associated with epileptiform activity. Patients with non-neoplastic forms of autoimmune encephalitis associated with AE-EA Group antibodies may have an underlying systemic autoimmune disorder or can develop symptoms following a viral infection or vaccination, but in many cases, no clear etiology is identified.

### Diagnosis and Treatment

Diagnosis of AE can be very challenging because it can present with a variety of neurologic and psychiatric manifestations that can be difficult to distinguish from other more common neuropsychiatric syndromes that cause behavioural disturbance.

The standard initial treatment commonly begins with high dose intravenous steroid regimens, generally methylprednisolone 1 mg/kg, followed by or given concurrently with either plasmapheresis or intravenous immunoglobulin (IVIg) administration. Such steroid regimens range from 3 to 5 days to chronic tapers until clinical stabilization is achieved. Plasma exchange and IVIg regimens generally run roughly 5 to 7 days. Intravenous rituximab may also be administered. Long term steroid-sparing immunosuppressants, such as azathioprine, may be used for maintenance therapy requiring continuous administration.

Recovery from AE is often long. In refractory cases or cases where there is a delay in treatment initiation, it can take several months or even years before patients can resume productive lives.

Cladribine or 2-chloro-2′-deoxyadenosine has been used in the oncology field with effects on lymphocytes. It has also been used in the treatment of multiple sclerosis (MS). (see U.S. Pat. No. 5,506,214), and it is currently used in the treatment of that condition. The therapeutic effect of cladribine on MS is, however, only evident three months after treatment. Cladribine has been tested as a possible treatment for the autoimmune neuromuscular disease myasthenia gravis. (see WO 2019/016505). Cladribine has also been reported to have been used in an attempt to treat a patient diagnosed with neuromyelitis optica. (see EP 3 099 307). Whilst cladribine has been used for treating other diseases including some leukemias and multiple sclerosis, and dosage regimens have been described (see EP 2263678) it could not have been predicted that cladribine would be effective in treating autoimmune encephalitis.

The inventor has unexpectedly found that cladribine may be beneficial in the treatment or amelioration of an autoimmune brain disorder, in particular autoimmune encephalitis. It was particularly beneficial in a patient that exhibited the symptoms of autoimmune encephalitis that was refractory to other forms of standard treatment. The inventor has further unexpectedly found that the sum of cladribine's effects on the immune system allows a relatively short period of treatment to provide beneficial effects on the disease for a prolonged period of over 10 months to two years without the need for frequent retreatment. Furthermore, the inventor has unexpectedly found that cladribine had a rapid effect on the severe symptoms of a patient suffering from refractory autoimmune encephalitis, compared with the therapeutic effect of cladribine on multiple sclerosis that is usually evident three months after treatment.

### SUMMARY OF THE INVENTION

According to one aspect of the invention there is provided 2-chloro-2′-deoxyadenosine, known as cladribine, or a pharmaceutically acceptable salt thereof, for use in the treatment or amelioration of autoimmune encephalitis, hereinafter referred to as AE, in a patient diagnosed with AE.

The cladribine may be beneficial for use where AE is refractory to usual standard treatments for AE.

The cladribine may be for use in the treatment of a patient known to have anti-brain antibodies, for example antibodies to the NMDA receptor.

It may also be for use in the treatment of a patient exhibiting the clinical picture indicative of an autoimmune encephalitis and with antibodies to another neuronal protein having been demonstrated, or where no specific neuronal antibodies have been detected.

According to a second aspect of the invention there is provided a pharmaceutical composition comprising 2-chloro-2′-deoxyadenosine, known as cladribine, for use in the treatment or amelioration of autoimmune encephalitis. The composition preferably comprises one or more pharmaceutically acceptable excipients.

The composition may comprise from 1 milligram (mg) to 30 mg of cladribine per unit dose, preferably from 5 mg to 30 mg, most preferably from 20 mg to 26 mg per unit dose if taken orally, or 8 mg to 12 mg if administered by injection.

Preferably the composition is to be administered orally. For oral administration the composition may be presented as a tablet, a capsule or a liquid formulation. It may also be presented in a liquid formulation suitable for injection.

Preferably the composition consists of cladribine or a pharmaceutically acceptable salt thereof.

According to another aspect of the invention there is provided use of 2-chloro-2′-deoxyadenosine (cladribine), or a pharmaceutically acceptable salt thereof, in the preparation of a medicament for the treatment or amelioration of autoimmune encephalitis.

Preferably the medicament is to be administered orally and is presented in the form of a tablet, capsule or liquid formulation.

An effective cumulative dose or amount of from 1 to 8.75 mg cladribine per kilogram of patient body weight (mg/kg) in the medicament may be taken, usually orally. Preferably the effective cumulative amount comprises from 1.5 mg/kg to 3.5 mg/kg of cladribine.

According to yet another aspect of the invention there is provided a method of treating or ameliorating autoimmune encephalitis in a subject suffering from the disease comprising administering to the subject, or patient, a pharmaceutical composition comprising an effective amount of 2-chloro-2′-deoxyadenosine (cladribine), or a pharmaceutically acceptable salt thereof.

The composition may be presented in unit dose form such as a tablet, capsule or liquid formulation for oral administration.

The pharmaceutical composition may be administered daily as a single dose.

### DETAILED DESCRIPTION OF THE INVENTION

### Definitions

“Amelioration” of a disease refers to the ability of a pharmaceutical composition or treatment to make the patient undertaking the treatment better or to improve the symptoms of the disease suffered by the patient or to make the disease more tolerable.

As used herein, “treating” or “treatment” means reducing, hindering the development of, controlling, alleviating and/or reversing the symptoms in an individual to which cladribine has been administered, as compared to the symptoms of an individual not being treated.

“Effective amount” of a composition refers to a composition which contains cladribine in an amount sufficient to provide a therapeutic dose over the course of treatment.

The term “unit dose” refers to physically discrete units suitable as unitary dosages for administration to patients, each such unit containing a predetermined quantity of cladribine calculated to produce the desired therapeutic effect in association with pharmaceutically acceptable ingredients.

The terms “effective cumulative amount” and “effective cumulative dose” refer to the total amount of cladribine given to a patient over time, i.e. the total dose of cladribine given in a series of treatments.

Cladribine and/or its pharmaceutically acceptable salts may be used in the practice of this invention. Suitable pharmaceutically acceptable salts refers to non-toxic acid addition salts that are generally prepared by reacting a compound with a suitable organic or inorganic acid. Examples of suitable salts include the hydrochloride, hydrobromide, sulphate, phosphate, citrate, acetate and maleate.

Cladribine may be prepared by processes well known in the art, such as those described in EP 173,059, U.S. Pat. No. 5,208,327 and Robins et al., J. Am. Chem. Soc., 106; 6379; (1984).

Whilst cladribine may be administered intravenously or subcutaneously, oral delivery is preferred for several reasons, the most important of which is patient compliance. There is also generally a cost benefit, since the cost of parenteral administration is much higher due to the necessity for the administration to be carried out by a doctor or nurse in a clinic, hospital or other specialised facility.

Oral administration of cladribine may be in capsule, tablet, oral suspension or syrup form, with capsules or tablets being preferred. Oral formulations of cladribine have been described in WO 2004/087100.

Pharmaceutical compositions of cladribine for use in the present invention may further comprise one or more pharmaceutically acceptable excipients such as alum, stabilizers, antimicrobial agents, buffers, colouring agents, flavouring agents, flavouring agents, adjuvants and the like. Where the composition is in the form of a tablet or capsule for oral administration conventional excipients, such as binding agents, fillers, lubricants, glidants, disintegrants and wetting agents may be included.

Binding agents include, but are not limited to, syrup, acacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulphate. Glidants include, but are not limited to, silicon dioxide.

Tablets or pills may be provided with an enteric layer in the form of an envelope that serves to resist disintegration in the stomach and permits the active ingredients to pass intact into the duodenum or to be delayed in release. A variety of materials can be used for enteric layers or coatings, including polymeric acids or mixtures of such acids with such materials as shellac, shellac and cetyl alcohol, cellulose acetate phthalate and the like.

Compositions of this invention may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminium stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

Treatments may be given as a number of courses, each course comprising for example five consecutive days of administration of one or two tablets or capsules containing 10 mg cladribine or drinking or infusing a similar amount of cladribine in a liquid formulation on each of five days. Patients suffering from AE may, for example, receive two such courses of treatment separated by several days, for example from 21 to 30 days, at the beginning of the treatment. This may be followed by two additional courses, also separated by from 21 to 30 days at the beginning of the second year of treatment, or only the first two courses may be used in a patient's therapy. Alternatively, a lower dose may be used and administered for from 5 to 10 consecutive days.

An advantage of using cladribine over the standard treatments of steroids and plasma exchange is that when treatment with cladribine is complete there is little or no risk of a relapse.

The invention will be further described with reference to the following examples:

### Example 1

### Powder in Capsule Formulation

Cladribine 10 mg
Microcrystalline cellulose 100 mg
Lactose 77.8 mg
Croscarmellose sodium 10 mg
Silicon dioxide 0.2 mg
Magnesium stearate 2 mg
Hard gelatin size 1 capsule shell

### Example 2

Treatment of a Person Suffering from Autoimmune-Mediated Limbic Encephalitis

A 38-year-old female was presenting with acute confusion, agitation and behavioural changes. Her past medical history was non-significant and no prior psychiatric or psychological problems were reported. Twelve months prior to the onset of illness she gave birth to healthy child. Upon admission, agitation and restlessness were observed, followed by progressive mutism and somnolence, which were the predominant symptoms of the disease within 5 days. The patient was administered standard antipsychotic treatment during her entire stay in the psychiatric ward, which included haloperidol, olanzapine, without any improvement in her psychiatric condition. On day 5, the patient's neurological condition significantly deteriorated; decreased level of consciousness and loss of muscle tone were observed. Involuntary movements of upper limbs, jaw and eyes were also noticed, as well as clonic seizures, which were treated with intravenous diazepam. The initial diagnosis was infectious encephalitis and the patient was transferred to a neurological ward at the state hospital, where cerebrospinal fluid (CSF) and blood samples were obtained and imaging studies of the brain, as well as EEG were performed.

Results of the CSF analysis revealed a lymphocytic pleocytosis (60 white cells/μl), a normal protein level (28.3 mg/dl; normal value range: 15-45 mg/dl) and a normal glucose level (81 mg/dl). EEG recordings revealed generalized rhythmic delta activity with superimposed rhythmic beta frequency activity (‘extreme delta brush’). Computed tomography (CT) of the head revealed no pathological changes. The magnetic resonance imaging (MRI), which was performed upon admission to the neurological ward, revealed only two hyperintense lesions in subcortical white matter in the frontal lobes on T2/FLAIR. Additionally, initial focal seizures evolved into generalized tonic-clonic repetitive seizures, which did not respond to various anticonvulsants, including clonazepam, sodium valproate, phenytoin and carbamazepine. Subsequently, treatment with acyclovir was initiated for presumptive viral encephalitis. Immunotherapy was also implemented and the patient received a daily dose of 20 g of IVIg for 5 consecutive days. The patient's condition deteriorated further; she developed a refractory status epilepticus with concomitant respiratory failure and autonomic instability and was referred to a neurologic ITU. She again had lumbar puncture and the blood and CSF samples were collected to search for neuronal surface antibodies and the anti-NMDAR antibodies were identified in the CSF (titers, 1:3.2), but not in the plasma. The diagnosis of anti-NMDAR encephalitis was made based on the above results.

After a further course of IVIg treatment her condition stabilised and started to improve over the next 3 weeks. Patient regained consciousness and started to breathe without support. She remained confused but she had short-term memory problems. Suddenly she again deteriorated and seizures and respiratory insufficiency appeared requiring ventilation. She received a course of steroids but without improvement.

The decision was made to give her a treatment trial of cladribine in the initial dose of 20 mg cladribine, administered by subcutaneous injection of 2.5 mg cladribine in 2.5 ml in each of four limbs (total 10 mg), on each of two consecutive days.

Over 3 consecutive weeks the condition of the patient gradually improved and she returned to normal neurological condition over subsequent 4 weeks. She reported no significant disability and was able to perform all usual activities. The only neurological sequel included parasomnia and complete loss of memory covering the entire hospitalization period. After extensive evaluation no apparent signs or laboratory results revealed evidence for neoplastic disease and no markers of tumor were found. The patient received 5 more maintenance courses of cladribine (20 mg over 2 consecutive days) with 2 months intervals. The follow up of 2 years did not show any recurrence of disease.

1.-31. (canceled)

32. A method of treating or ameliorating autoimmune encephalitis in a patient diagnosed with autoimmune encephalitis (AE) comprising administering to the subject a pharmaceutical composition comprising an effective amount of 2-chloro-2′-deoxyadenosine, hereinafter referred to as cladribine, or a pharmaceutically acceptable salt thereof or a composition comprising cladribine, or a pharmaceutically acceptable salt thereof and a pharmaceutically acceptable excipient.

33. The method according to claim 32, wherein the AE is refractory to standard treatments.

34. The method according to claim 32, wherein the patient is known to have anti-brain antibodies.

35. The method according to claim 32, wherein the patient is known to have antibodies to the NMDA receptor

36. The method according to claim 32, wherein the patient is known to have autoimmune encephalitis.

37. The method according to claim 32, wherein the composition is administered in unit dose form.

38. The method according to claim 37, wherein the unit dose form is a tablet, a capsule or liquid formulation.

39. The method according to claim 32, wherein the effective amount of cladribine is administered orally.

40. The method according to claim 39, wherein the composition is administered daily as a single dose.

41. The method according to claim 37, said unit dose form comprising from 1 mg to 30 mg of cladribine or salt thereof per unit dose

42. The method according to claim 41, said unit dose form comprising 5 mg to 30 mg of cladribine or salt thereof per unit dose.

43. The method according to claim 41, said unit dose form comprising 20 mg to 26 mg of cladribine or salt thereof per unit dose.

44. The method according to claim 41, said unit dose form comprising 8 mg to 12 mg of cladribine or salt thereof per unit dose.

45. The method according to claim 41, said unit dose form comprising 10 mg of cladribine or salt thereof per unit dose.
